(19) United States
(12) Patent Application Publication
(10) Pub. No.: US 2010/0119633 A1
(43) Pub. Date: May 13, 2010

(54) Analgesic oil containing plant based extracts from solar infusions

(76) Inventor: Desiree Astorino McCloskey, Fresno, CA (US)

Correspondence Address: MICHAEL A. GUTH, 2-2905 EAST CLIFF DRIVE, SANTA CRUZ, CA, 95062, US

(21) Appl. No.: 12/291,673

(22) Filed: Nov. 12, 2008

(51) Int. Cl. A61K 36/28; A61P 29/00

(52) U.S. Cl. 424/764

(57) ABSTRACT

A liquid analgesic composition and method for its preparation and use. The composition may be prepared using plant extracts obtained by solar infusion. The composition may include extracts from dandelion, arnica, and chamomile, as well as other essential oils.

### BACKGROUND

1. Field of the Invention

The present invention relates to health care products, and more specifically to a composition of a liquid analgesic produced from extracts of plants.

2. Background

Pain is an unpleasant sensory experience which may occur for many reasons, and can be associated with tissue damage. Nerve endings in the painful area send impulses to the brain which result in the sensation of pain. Pain may be the result of a recent injury, or of an older injury that has not properly healed. Pain may be the result of long term stress on the body from work or poor posture.

Relief from pain is sought after by those experiencing it. A variety of therapies may be used. Many people use prescription or over the counter oral medications to address their pain. The risk of narcotic addiction with prescription medications and the side effects, sometimes dangerous, of over the counter medications have turned many people to herbal therapies.

### SUMMARY

A liquid analgesic composition and method for its preparation and use. The composition may be prepared using plant extracts obtained by solar infusion. The composition may include extracts from dandelion, arnica, and chamomile, as well as other essential oils.

### BRIEF DESCRIPTION OF THE DRAWINGS

FIG. 1 is a flow chart illustrating a process of producing a plant extract using solar infusion.

FIG. 2 is a flow chart illustrating a process for producing an analgesic composition according to some embodiments of the present invention.

### DETAILED DESCRIPTION

Grapeseed oil is a vegetable oil typically pressed from the seeds of various varieties of grapes. Solvent extraction methods may be used to produce grapeseed oil as well. Typically, grapeseed oil refers to oil pressed from the seeds of the Vitis vinifera grapes. Grapeseed oil typically contains linoleic acid, oleic acid, palmitic acid, stearic acid, and palmitoleic acid.

Solar infusion is a method of extracting constituents of an item, typically an organic plant based material. Solar infusion may comprise placing organic matter into liquid and having the liquid/matter mix receive solar radiation. An example could be putting the liquid and matter into a clear glass jar, and placing the jar into the sun. Constituents of the matter are absorbed by the liquid, or otherwise enter the liquid. The liquid/matter mix may be filtered to yield an extract of the organic matter. The extract is viewed as the combination of the original liquid and matter entered into the liquid during the process.

In some embodiments of the present invention, extracts are created from plant matter. The plant matter may be carefully grown to be relatively free of herbicides or other chemicals. The plant matter may be organic. In some embodiments, the plant matter used during the solar infusion process is flowers of plants.

In some embodiments, the liquid used during the solar infusion process is a vegetable oil. The oil may be grapeseed oil. In some embodiments of the present invention, a process to create an extract by solar infusion may be as follows. The plant matter used may be freshly harvested. The plant matter used may be predominantly the flowers of the plants. The flowers may be washed and then dried. A ratio of 2 ounces of plant material to 500 cc of grapeseed oil may be used. The combination of plant material and the grapeseed oil may be placed into sterilized glass jars and sealed.

The glass jars may be placed into the sun. In some embodiments, the jars are placed into the sun for a minimum of 3 weeks during summer months to allow for sufficient infusion. The bottles may be placed in an orientation to maximize the amount of direct sunlight received. After the completion of the sun portion of the process, the liquid/flower mix may be filtered using a fine mesh screen. The filtered liquid is referred to as the extract of this flower (or other material, if used).

Essential oil is a term typically used to describe a concentrated liquid containing volatile compounds from plants, including aroma compounds. Typically, essential oils are created using a distillation process.

The common name dandelion is the common name given to flowering plants in the genus taraxacum in the family Asteraceae. Two of the species, officinale and erytrhospermum, are found worldwide. The dandelion has a history of being used for healing purposes, but typically it is the root that is used. Also, the leaves have been used for tea and for other reasons, including those relating to health.

There are a number of species of chamomile. German chamomile, also known as wild chamomile, is known botanically as Matricaria chamomilla. The flowers of German chamomile are known to have a very bitter taste. German chamomile is commonly used in chamomile tea. It has some history of use as a nerve sedative, and there is some history of it being used internally for calming of the digestive tract.

Arnica (Arnica Montana), also known as Leopard's Bane, is a perennial herb and is indigenous to central Europe. Studies on the effects of arnica preparations have demonstrated antimicrobial, anti-inflammatory, respiratory-stimulant, positive inotropic, and tonus increasing actions. The anti-inflammatory effects are attributed to helenalin, whose actions include a marked antiedemic effect that has been confirmed in experimental models.

In some embodiments of the present invention, an oil containing plant based extracts is prepared for use a topical analgesic and for therapeutic use with muscle tightness, pain, bruises, strains, arthritis relief, and for other related needs. A combination of arnica, German chamomile, and dandelion, and in some embodiments with essential oils of rosemary and lavender, is used to produce a topical therapeutic oil.

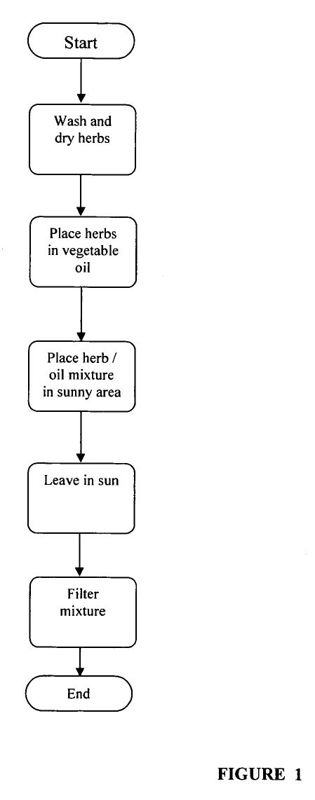

In some embodiments of the present invention, as seen in FIG. 1, a process of solar infusion is used to prepare plant extracts. An example of the process for making an extract from dandelion (Taraxacum officianale), from German chamomile, and from arnica begins with the harvesting of the fresh flowers of these herbs. The plant extracts may be made separately, although using a similar process for each herb. To obtain fresh flowers, the herbs are first grown and allowed to flower. Then the fresh flowers are harvested. The fresh flowers of the herbs may be gently washed and dried. The process of solar infusion is used to extract material from the flowers of the herbs into a vegetable oil. In some embodiments of the present invention, pure grapeseed oil is used.

The flowers of the herbs will be immersed into the grapeseed oil, and this will typically be done with each type of herb being processed separately. A ratio of 2 ounces of plant material to 500 cc of grapeseed oil may be used. The combination of plant material and the grapeseed oil may be placed into sterilized glass jars and sealed.

The glass jars may be placed into the sun. In some embodiments, the jars are placed into the sun for a minimum of 3 weeks during summer months to allow for sufficient infusion. The bottles may be placed in an orientation to maximize the amount of direct sunlight received. After the completion of the sun portion of the process, the liquid/flower mix may be filtered using a fine mesh screen. The filtered liquid is referred to as the extract of this flower (or other material, if used).

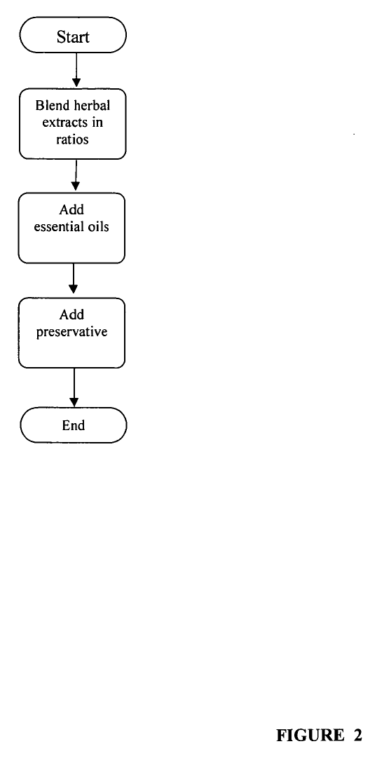

After completion of the solar infusion process for each of the herbal flower types, the different extracts will be blended together, as seen in FIG. 2. The ratio of these different extracts has been derived from experimentation, which included different ratios of the herbs mentioned above as well as other herbs. The ratios and range of rations described herein has been found to be especially effective in the treatment of muscle tightness, pain, bruises, strains, arthritis relief, although the use of compounds according to embodiments of the present invention is not limited to treatment of only these conditions. A contra-indication for use with oils described herein is application to broken, incised, or burned skin.

After completion of the creating the extracts of the herbs using solar infusion, the dandelion extract, the arnica extract, and the German chamomile extract are mixed in preferred ratios. In some embodiments, the amount of the selected extracts used a volume percentage of the final product is 50-75% dandelion extract, 10-30% arnica extract, and 5-15% German chamomile extract. In some embodiments, the amount of the selected extracts used as a volume percentage of the final product is 65% dandelion extract, 20% arnica extract, and 10% German chamomile. In such an embodiment, wherein the total volume percentage of the herbal extracts is less than 100%, essential oils may be added to the composition. In such a case, the herbal extracts may be blended in a 65/20/10 volumetric ratio for dandelion, arnica, and German chamomile, respectively.

In some embodiments of the present invention, essential oils of lavender and rosemary are added to the composition. In some embodiments, the volume percentage of each of the lavender and rosemary oils is in the range of 1-6%. In some embodiments, the volume percentage of the final product for lavender oil is 2.5%, and for rosemary oil is 2.5%. It should be noted that essential oil of lavender is also referred to as lavender oil, and that the essential oil of rosemary is also referred to as rosemary oil.

In some embodiments, it may be preferable to add a material to the final composition as a preservative. Grapefruit seed extract may be used. For example, in a small bottle of prepared composition, 3 drops of grapefruit seed extract may be added.

In some embodiments of the present invention, a method of therapeutic treatment for muscle tightness, pain, bruises, strains, and/or arthritis relief includes the topical use of a composition as described herein. A small amount, an example being five drops, of compositions as described herein may be applied to the affected area and rubbed into the skin. This application may be repeated after thirty minutes.

As evident from the above description, a wide variety of embodiments may be configured from the description given herein and additional advantages and modifications will readily occur to those skilled in the art. The invention in its broader aspects is, therefore, not limited to the specific details and illustrative examples shown and described. Accordingly, departures from such details may be made without departing from the spirit or scope of the applicant's general invention.

What is claimed is:

1. An herbal composition useful as a topical analgesic, said composition comprising:
an extract of dandelion flowers;
an extract of arnica; and
an extract of German chamomile.

2. The composition of claim 1 further comprising lavender oil.

3. The composition of claim 2 further comprising rosemary oil.

4. The composition of claim 1 wherein said extract of dandelion flowers comprises 50-70% by volume of said composition.

5. The composition of claim 4 wherein said extract of arnica comprises 10-30% by volume of said composition.

6. The composition of claim 5 wherein said extract of German chamomile comprises 10-20% by volume of said composition.

7. The composition of claim 2 wherein said extract of dandelion flowers comprises 50-70% by volume of said composition, and wherein said extract of arnica comprises 10-30% by volume of said composition, and wherein said extract of German chamomile comprises 10-20% by volume of said composition, and wherein said lavender oil comprises 1-6% by volume of said composition.

8. The composition of claim 3 wherein said extract of dandelion flowers comprises 50-75% by volume of said composition, and wherein said extract of arnica comprises 10-30% by volume of said composition, and wherein said extract of German chamomile comprises 10-20% by volume of said composition, and wherein said lavender oil comprises 1-6% by volume of said composition, and wherein said rosemary oil comprises 1-6% by volume of said composition.

9. The composition of claim 1 wherein said extract of dandelion flowers comprises 65% by volume of said composition, and wherein said extract of arnica comprises 20% by volume of said composition, and wherein said extract of German chamomile comprises 15% by volume of said composition.

10. The composition of claim 3 wherein said extract of dandelion flowers comprises 65% by volume of said composition, and wherein said extract of arnica comprises 20% by volume of said composition, and wherein said extract of German chamomile comprises 15% by volume of said composition, and wherein said lavender oil comprises 2.5% by volume of said composition, and wherein said rosemary oil comprises 2.5% by volume of said composition.

11. The composition of claim 1 wherein said extracts are oil based extracts.

12. The composition of claim 11 wherein said extracts are grapeseed oil based extracts.

13. The composition of claim 3 wherein said extracts are grapeseed oil based extracts.

14. The composition of claim 8 wherein said extracts are oil based extracts.

15. The composition of claim 14 wherein said extracts are grapeseed oil based extracts.

16. The composition of claim 10 wherein said extracts are oil based extracts.

17. The composition of claim 16 wherein said extracts are grapeseed oil based extracts.

18. The composition of claim 15 wherein said grapeseed oil based extracts are produced by the process of solar infusion.

19. The composition of claim 17 wherein said grapeseed oil based extracts are produced by the process of solar infusion.
